(19) United States
(12) Patent Application Publication
(10) Pub. No.: US 2005/0112645 A1
(43) Pub. Date: May 26, 2005

(54) Detecting unit for detecting hybridization and other interactions and DNA chip and other bioassay substrates having the detecting unit

(76) Inventors: Yuji Segawa, Tokyo (JP); Takayoshi Mamine, Kanagawa (JP); Oliver Harnack, Stuttgart (DE)

Correspondence Address: FROMMER LAWRENCE & HAUG LLP, 745 FIFTH AVENUE, NEW YORK, NY, 10151, US

(21) Appl. No.: 10/955,280

(22) Filed: Sep. 30, 2004

(30) Foreign Application Priority Data
Oct. 2, 2003 (JP) 2003-344861

(51) Int. Cl. C12Q 1/68; C12M 1/34

(52) U.S. Cl. 435/6; 435/287.2; 205/777.5

(57) ABSTRACT

A detecting unit for detecting interaction between substances includes a pair of first opposed electrodes disposed opposite to each other so as to sandwich a reaction area providing a field for the interaction between the substances, and both or one of electrodes forming second opposed electrodes disposed opposite to each other in a direction of an axis crossing an opposing axis of the first opposed electrodes.

### BACKGROUND OF THE INVENTION

The present invention relates to a technique for increasing accuracy of detection of interaction between substances by using effects of an electric field, and particularly to a technique for adjustment, movement, and fixation of higher order structures of the substances, removal of undesired substances, and the like by applying a predetermined electric field with electrodes provided in a reaction field for the interaction between the substances.

Main background arts relating to the present invention will be described. A first background art (related art) is a technology relating to an integrated substrate for a bioassay referred to as a so-called DNA chip or DNA micro-array (hereinafter referred to generically as a “DNA chip”) on which predetermined DNAs are finely arranged by micro-array technology. This DNA chip technology is characterized in that comprehensive analysis of interaction between molecules such as hybridization or the like is possible because a wide variety and a large number of DNA oligo chains, cDNAs (complementary DNAs) or the likes are integrated on a glass substrate or a silicon substrate. Therefore the DNA chip is used for analysis of gene mutation, analysis of SNPs (Single Nucleotide Polymorphism), analysis of frequency of gene expression, and the like, and is beginning to come into wide use in development of new drugs, clinical diagnosis, pharmacological genomics, forensic medicine and other fields. In addition to DNA chips, protein chips having protein fixed on a substrate, biosensor chips for analyzing interaction between various substances, and the like have been developed.

A second background art is a technology relating to action of an electric field on a substance present in a charged state in a liquid phase. Specifically, a nucleotide chain (nucleic acid molecule) is known to be stretched or moved under the action of an electric field in a liquid phase. According to its principles, phosphate anions (negative charge), which form a frame of the nucleotide chain, and hydrogen atoms (positive charge) resulting from ionization of water around the phosphate anions are considered to together form an ion cloud. A polarization vector (dipole) generated by the negative charge and the positive charge points in one direction as a whole as a result of application of a high-frequency high voltage, and consequently the nucleotide chain is stretched. In addition, when a non-uniform electric field in which lines of electric force concentrate at one part is applied, the nucleotide chain moves to the part where the lines of electric force concentrate (see Non-Patent Literature 1). When a DNA solution is placed in a gap of a few ten to a few hundred μm between micro-electrodes and a high-frequency electric field of about 1 MV/m and about 1 MHz is applied to the DNA solution, dielectric polarization occurs in DNA present in a form of a random coil. As a result, the DNA molecule is stretched into a form of a straight line in parallel with the electric field. It is known that due to this electrodynamic effect referred to as “dielectrophoresis,” the polarized DNA spontaneously draws to an electrode edge, and is then fixed on the electrode edge with one end of the DNA in contact with the electrode edge (see Non-Patent Literature 2).

[Non-Patent Literature 1] Seiichi Suzuki, Takeshi Yamanashi, Shin-ichi Tazawa, Osamu Kurosawa, and Masao Washizu: “Quantitative analysis on electrostatic orientation of DNA in stationary AC electric field using fluorescence anisotropy,” IEEE Transaction on Industrial Applications, Vol. 34, No. 1, pp. 75 to 83 (1998)

[Non-Patent Literature 2] Masao Washizu, “DNA handling while viewing,” Visualization Information, Vol. 20, No. 76 (January 2000)

The above-described DNA chip technology sets a reaction area providing a field for interaction between substances in a liquid phase on a substrate in advance, and fixes a nucleotide chain for detection such as probe DNA or the like in the reaction area, to analyze hybridization as interaction between the nucleotide chain for detection and a target nucleotide chain complementary to the nucleotide chain for detection. However, poor efficiency of the hybridization presents important technical problems in that a long time is required for the reaction and in that detection accuracy is low because of occurrence of false positives and false negatives.

In implementing this DNA chip technology, if the nucleotide chain for detection present with a terminal portion thereof fixed in the reaction area can be adjusted to a stretched state, it is considered possible to eliminate steric hindrance caused by a higher order structure in a random coil form of the molecule and hindrance caused by interference (for example adhesion or contact) between the nucleotide chain for detection and a peripheral surface, and consequently improve the hybridization efficiency. It is also considered that if substances other than normal complementary chains can be removed from a part for detection, the detection accuracy can be increased.

### SUMMARY OF THE INVENTION

It is accordingly an object of the present invention to provide a detecting unit and a fundamental structure of a bioassay substrate that make it possible to freely adjust, move, and fix higher order structures of substances, and remove undesired substances, for example, by applying a predetermined electric field with opposed electrodes provided in a reaction field.

According to the present invention, there is provided a detecting unit for detecting interaction between substances, the detecting unit including: a pair of first opposed electrodes disposed opposite to each other so as to sandwich a reaction area providing a field for the interaction between the substances; and both or one of electrodes forming second opposed electrodes disposed opposite to each other in a direction of an axis crossing an opposing axis of the first opposed electrodes. There is also provided a bioassay substrate including a DNA chip characterized by having the interaction detecting unit.

In the thus formed interaction detecting unit, the “first opposed electrodes” are a pair of electrodes disposed so as to sandwich the reaction area and arranged so as to form an opposed relation to each other. The “second opposed electrodes” have one of the following configurations described in (1) and (2): (1) a configuration of electrode arrangement in which both the electrodes are disposed opposite to each other so as to sandwich the reaction area and the opposing axis connecting the electrodes to each other crosses the opposing axis of the first opposed electrodes; (2) a configuration of electrode arrangement in which only one of the second opposed electrodes is provided in the detecting unit and the other electrode of the pair is an external electrode. The external electrode refers to an electrode formed on a member or area separate from substrate material of a substrate or the like where the first opposed electrodes and the reaction area are formed.

As to the above-mentioned “second opposed electrodes,” when the configuration of electrode arrangement (2) is employed, for example, the opposing axis connecting the second opposed electrodes to each other can be perpendicular to a surface on which a substance for detection is fixed in the reaction area. Incidentally, when the reaction area is disposed horizontally, the opposing axis connecting the second opposed electrodes to each other is vertical.

Next, the present invention provides a constitution in which surfaces of electrodes forming the first opposed electrodes and the second opposed electrodes are covered with an insulating layer. This insulating layer can be formed by a material selected from SiO₂, SiN, SiOC, SiC, SiOF, and TiO₂, for example. It is also possible to adopt a constitution in which at least one of the second opposed electrodes is formed by a transparent conductor. The transparent conductor has an advantage of being able to form an electrode that transmits exciting light for detection.

As an “electric field” used in the present invention, an “alternating-current electric field” is most suitable because the alternating-current electric field can suppress heat generation and gas generation by electrolysis. Hence, the first opposed electrodes and the second opposed electrodes are both electrodes for application of an alternating-current electric field. Further, an area of one of the second opposed electrodes is made smaller than an area of the other opposite electrode, or a surface of at least one of the second opposed electrodes is processed into a rough surface, for example formed by patterning with insular shapes, whereby lines of electric force are concentrated on the electrode having the smaller area or the electrode processed into a rough surface or formed by patterning with insular shapes, thus forming a so-called “non-uniform electric field” in the reaction area.

The present invention can use the “first opposed electrodes” as means for drawing free substance and/or substance showing false interaction that are present in the reaction area to each electrode side by the electric field, and use the “second opposed electrodes” as means for elongating a substance for detection fixed in the reaction area by the electric field.

The use of the former means has an advantage of eliminating a need for so-called washing work performed by flowing a predetermined aqueous solution in the reaction area after progress of interaction in the reaction area. The use of the latter means can adjust a higher order structure of the substance for detection to a stretched structure that facilitates the progress of the interaction and is free from “steric hindrance.” For example, a nucleic acid coiled in the form of a random coil can be adjusted to a stretched structure in a form of a straight chain with a base portion exposed.

The present invention develops the means to provide means for fixing nucleic acid as substance drawn by effect of the electric field to electrodes forming the first opposed electrodes on surfaces of the electrodes via one of an avidin-biotin bond and a disulfide bond (—S—S— bond).

The present invention also provides means for trapping, by a double stranded nucleic acid, a surplus intercalator as substance drawn to the electrodes forming the first opposed electrodes, the surplus intercalator being made to be present in the vicinity of the surfaces of the electrodes, and fixing the double stranded nucleic acid on the surfaces of the electrodes via one of an avidin-biotin bond and a disulfide bond.

Further, the present invention provides means for making gels including cations and anions present in advance on the surfaces of the electrodes forming the first opposed electrodes, and electrostatically bonding a negatively charged nucleic acid and a positively charged surplus intercalator as substances drawn to each of the first opposed electrodes to the cations and the anions, respectively.

A fundamental constitution of a detecting unit that implements the means as described above at least includes: opposed electrodes (for example second opposed electrodes of the constitution) for elongating nucleic acid as a complementary chain for hybridization; and an electrode (for example first opposed electrodes of the constitution) used for dissociating and removing free nucleic acid present in a field of the hybridization and/or nucleic acid showing mis-hybridization. The detecting unit having such a constitution can be used as a hybridization detecting unit.

The hybridization detecting unit generally functions as a part for detecting hybridization progressing between nucleic acid for detection (for example probe DNA) present in a state of being fixed in a reaction area formed on a substrate or the like or in a free state and target nucleic acid later dropped into the reaction area.

In such a detecting unit, means can be provided so that a single stranded nucleic acid having a base sequence complementary to a base sequence specific to the target nucleic acid or a base sequence modified at a terminal of the target nucleic acid is made to be present on surfaces of electrodes forming the first opposed electrodes, and surplus target nucleic acid is trapped on the surfaces of the electrodes by hybridization between the single stranded nucleic acid and the surplus target nucleic acid. The detecting unit can also provide means for trapping a surplus intercalator in a double stranded nucleic acid obtained by the hybridization or a double stranded nucleic acid added separately.

The detecting unit can also provide means for decomposing substance drawn to the electrodes forming the first opposed electrodes by covering the surfaces of the electrodes forming the first opposed electrodes with an insulating layer formed of TiO₂, irradiating the insulating layer with ultraviolet light, and thereby making the TiO₂ function as a catalyst.

Main technical terms used in the present invention will be defined in the following. First, “interaction” used in the present invention widely refers to chemical bonds including non-covalent bonds, covalent bonds, and hydrogen bonds between substances, or dissociations, and includes hybridization, which is a complementary bond between nucleic acids (nucleotide chains), for example.

Next, “opposed electrodes” refer to at least one pair of electrodes disposed such that surfaces of the electrodes are opposed to each other. An “opposing axis” refers to an axis formed by a straight line connecting centers of surfaces of two opposed electrodes to each other. “Crossing” includes both crossing on an identical plane with a point of intersection formed, and three-dimensional crossing without formation of a point of intersection. As for a crossing angle, angles other than a right angle can be used as long as objects and effects of the present invention are achieved.

“Nucleic acid” in the present application refers to a polymer of phosphoric ester of nucleosides resulting from glycosidic linkage between a purine or pyrimidine base and sugar, and widely includes DNA (entire length or a fragment thereof) obtained by polymerizing oligonucleotides, polynucleotides, purine nucleotides, and pyrimidine nucleotides, including probe DNA, cDNA (c probe DNA) obtained by reverse transcription, RNA, polyamide nucleotide derivatives (PNA) and the like.

“Hybridization” refers to reaction for forming complementary chains (a double strand) between nucleotide chains having complementary base sequence structures. “Mis-hybridization” refers to the complementary chain forming reaction that is not normal.

A “reaction area” is an area that can provide a reaction field for interaction such as hybridization or the like, and includes a reaction field having a well shape that can store a liquid phase or gel, for example. The interaction occurring in the reaction area is not narrowly limited as long as objects and effects of the present invention are achieved. For example, it is possible to effect not only interaction between single stranded nucleic acids, that is, hybridization, but also interaction between peptide (or protein) and desired double stranded nucleic acid formed from nucleic acid for detection, enzyme response reaction, and other intermolecular interactions. When the double stranded nucleic acid is used, for example, it is possible to analyze for example a bond between a receptor molecule such as a hormone receptor as a transcription factor and a response element DNA portion.

“Free substance” in a reaction area includes for example a surplus of target nucleic acid having a base sequence portion complementary to a nucleic acid for detection such as probe DNA or the like present in the reaction area, and a surplus of so-called “intercalators” having a characteristic of being inserted into and bonded to complementary chains obtained as a result of hybridization.

“Steric hindrance” refers to a phenomenon in which desired reaction (hybridization in the case of the present application) does not occur easily because presence of a bulky substituent in the vicinity of a center of reaction within a molecule or a position or three-dimensional structure (higher order structure) of the reacting molecule makes it difficult for a molecule to which the reaction is to occur to approach.

“Dielectrophoresis” is a phenomenon in which a molecule is driven to a stronger electric field in a non-uniform electric field. Even when an alternating-current voltage is applied, the same driving effects as in a case of a direct-current voltage can be obtained because polarization polarity is reversed with reversal of polarity of the voltage applied (see Teru Hayashi (editor), “Micromachines and Materials Engineering (published by CMC),” pp. 37 to 46, Chapter 5 “Manipulation of Cells and DNA”).

A “bioassay substrate” refers to an information integrated substrate used for purposes of biochemical or molecular biological analyses, and includes a so-called DNA chip.

According to the present invention, by providing opposed electrodes having opposing axes crossing each other such that the opposed electrodes face a reaction area, and applying a predetermined voltage to these opposed electrodes in predetermined timing, it is possible to freely adjust the higher order structure of substance such as nucleic acid present in the reaction area, move the substance along an electric field, fix a terminal portion of the substance to an electrode surface, and dissociate and remove undesired substance that causes detection errors, for example.

Specifically, on a surface part of a bioassay substrate such as a DNA chip or the like, two pairs of opposed electrodes are disposed in a relation of arrangement of opposing axes crossing each other in a reaction area that provides a field for interaction such as hybridization or the like, and target substance such as target DNA or the like is drawn to the vicinity of one selected electrode surface by effect of an alternating-current electric field formed between these opposed electrodes, whereby a time for the interaction can be shortened.

By the effect of the applied electric field, it is possible to adjust probe DNA and target DNA to a stretched state. By preventing steric hindrance between DNAs, it is possible to improve hybridization efficiency and prevent occurrence of false positives or false negatives, for example.

By forming an alternating-current electric field using the other pair of electrodes in the reaction area, and collecting surplus intercalators and surplus DNA on surfaces of the electrodes, it is possible to reduce noise in the hybridization detecting unit and thus obtain a signal with a good S/N. That is, accuracy of detection of hybridization can be improved.

By elongating substance for detection such as probe DNA or the like and fixing the substance for detection in a state of being aligned on the electrode surface by the electric field using one pair of opposed electrodes, it is possible to efficiently collect (remove) the surplus DNA and the surplus intercalators using the other pair of opposed electrodes. That is, since the electrode surface on which the probe DNA or the like is fixed can be adjusted to an ordered state by combining both the elongating effect and the aligning and fixing effect of the electric field, the surplus DNA and the surplus intercalators present on the electrode surface can be quickly moved to be collected or removed from a detection area.

### BRIEF DESCRIPTION OF THE DRAWINGS

FIG. 1 is a plan view for schematically illustrating a concept of fundamental structure of a detecting unit according to the present invention;

FIG. 2 is a sectional view taken along a vertical direction for schematically illustrating a concept of fundamental structure of a detecting unit according to the present invention;

FIG. 3 is a perspective external view showing main parts of the detecting unit three-dimensionally;

FIG. 4 is a diagram schematically showing an example of a procedure for turning on/off switches;

FIG. 5 is a diagram schematically showing a modified example of the procedure;

FIG. 6 is a diagram schematically showing a state in which surplus target DNA and surplus intercalators are being drawn to an electrode and an electrode forming first opposed electrodes, taking the detecting unit as an example;

FIG. 7 is a diagram showing a structure of one concrete example of a detecting unit according to the present invention;

FIG. 8 is a diagram showing a structure of one concrete example of a detecting unit according to the present invention;

FIG. 9 is a diagram showing a structure of one concrete example of a detecting unit according to the present invention;

FIG. 10 is a diagram showing a structure of a detecting unit according to the present invention;

FIG. 11 is a diagram showing a structure of a detecting unit using dummy double stranded DNA;

FIG. 12 is a diagram showing a structure of a detecting unit using gel including cations and gel including anions; and

FIG. 13 is a diagram showing an example of a substrate in a form of a disc on which detecting units according to the present invention are disposed.

### DETAILED DESCRIPTION OF PREFERRED EMBODIMENTS

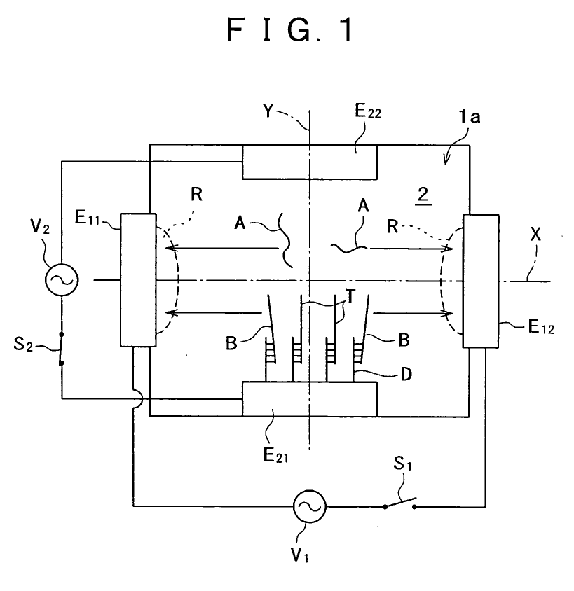

Preferred embodiments of the present invention will hereinafter be described with reference to the accompanying drawings. FIG. 1 is a plan view for schematically illustrating a concept of a first fundamental structure of a detecting unit for detecting interaction between substances (hereinafter abbreviated to a “detecting unit”) according to the present invention.

Reference 1a in FIG. 1 denotes the detecting unit having the first fundamental structure. The detecting unit 1a is formed on for example a substrate formed of glass, synthetic resin, or the like. The detecting unit 1a is a part devised to detect interaction between substances. A reaction area 2 that can store a liquid phase serving as a reaction field or retain gel or the like similarly serving as a reaction field is formed in the detecting unit 1a.

FIG. 1 is a view of the reaction area 2 and a structure on the periphery of the reaction area 2 as viewed from above. As shown in FIG. 1, two pairs of opposed electrodes are formed in the reaction area 2 of the detecting unit 1a.

Specifically, first opposed electrodes E₁₁ and E₁₂ disposed on a right and a left facing the drawing, and second opposed electrodes E₂₁ and E₂₂ formed on an opposing axis Y crossing an opposing axis X that connects the first opposed electrodes E₁₁ and E₁₂ with each other are provided so as to sandwich the reaction area 2. That is, the detecting unit 1a has the first opposed electrodes E₁₁ and E₁₂ and the second opposed electrodes E₂₁ and E₂₂ formed in an X-Y plane.

The first opposed electrodes E₁₁ and E₁₂ are connected to an alternating-current power supply V₁. The first opposed electrodes E₁₁ and E₁₂ are formed to apply a high-frequency alternating-current electric field to the reaction area 2 in response to on/off operation of a switch S₁. The second opposed electrodes E₂₁ and E₂₂ are connected to an alternating-current power supply V₂. The first opposed electrodes E₁₁ and E₁₂ are formed to apply a high-frequency alternating-current electric field to the reaction area 2 in response to on/off operation of a switch S₂. (The same is true for detecting units of other embodiments to be described later. This description will hereinafter be omitted.)

It is desirable that a surface of each of the first opposed electrodes E₁₁ and E₁₂ and the second opposed electrodes E₂₁ and E₂₂ be covered with an insulating layer formed by a material selected from SiO₂, SiN, SiOC, SiC, SiOF, and TiO₂. (The same is true for the detecting units of the other embodiments to be described later. This description will hereinafter be omitted.) This measure is to prevent electrochemical reaction by an ionic solution that may be stored in the reaction area 2.

The first opposed electrodes E₁₁ and E₁₂ play a role of drawing a free substance A and a substance B showing false interaction that are present in an area between the second opposed electrodes E₂₁ and E₂₂ and which can cause decrease in accuracy of detection and decrease in efficiency of interaction to sides of the first opposed electrodes E₁₁ and E₁₂ by electrodynamic force of dielectrophoresis. Areas R and R on a periphery of the electrodes E₁₁ and E₁₂ are fields where the free substance A and the false interaction substance B are removed from a field of interaction and accumulated. (The same is true for the detecting units of the other embodiments to be described later. This description will hereinafter be omitted.)

The second opposed electrodes E₂₁ and E₂₂ mainly play a role of elongating a detection substance D fixed on a surface of one electrode (E₂₁ in this case) along a direction of an electric field by electrodynamic force of dielectrophoresis and play a role of moving a target substance T showing specific interaction with the detection substance D along the electric field. (The same is true for the detecting units of the other embodiments to be described later.)

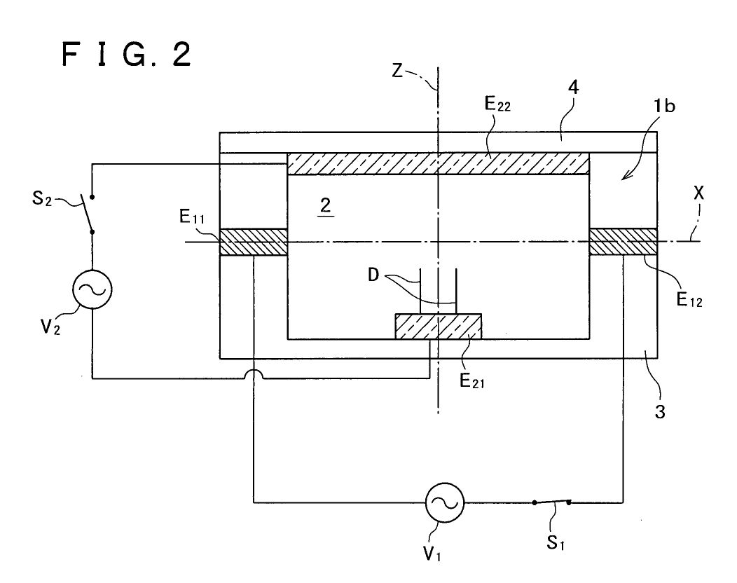

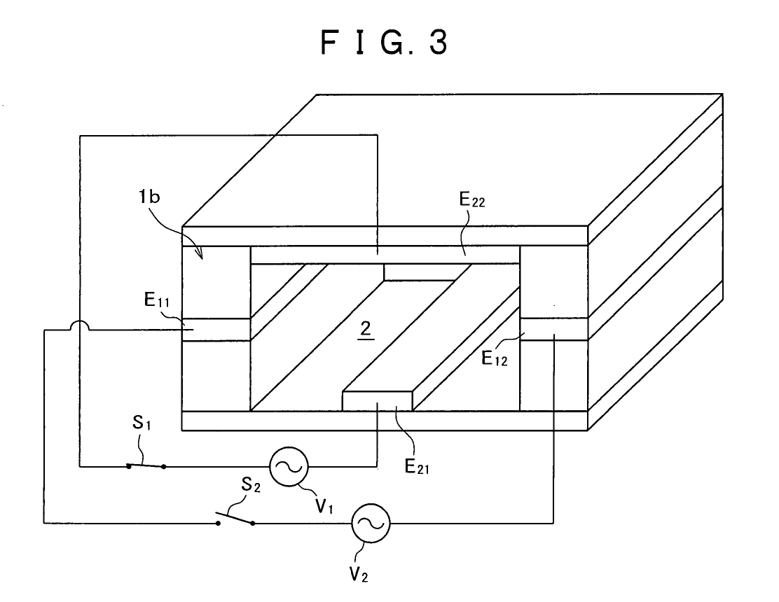

FIG. 2 is a sectional view taken along a vertical direction for schematically illustrating a concept of a second fundamental structure of a detecting unit according to the present invention. FIG. 3 is a perspective external view showing main parts of the detecting unit 1b three-dimensionally. Reference 1b shown in FIG. 2 and FIG. 3 denotes the detecting unit having the second fundamental structure.

As with the detecting unit 1a, the detecting unit 1b is formed on for example a substrate (denoted by reference numeral 3 in the figures) formed of glass, synthetic resin, or the like. The detecting unit 1b is a part devised to detect interaction between substances. A reaction area 2 that can store a liquid phase serving as a reaction field or retain gel or the like similarly serving as a reaction field is also formed in the detecting unit 1b.

Also in the detecting unit 1b, first opposed electrodes E₁₁ and E₁₂ disposed on a right and a left facing the drawing, and second opposed electrodes E₂₁ and E₂₂ formed on an opposing axis Z crossing an opposing axis X that connects the first opposed electrodes E₁₁ and E₁₂ with each other are provided so as to sandwich the reaction area 2. The detecting unit 1b is different from the detecting unit 1a in that the opposing axis Z is perpendicular to a surface on which a detection substance is fixed in the reaction area 2.

In either case, the two pairs of the opposed electrodes E₁₁ and E₁₂ and E₂₁ and E₂₂ formed in the detecting unit 1a and the detecting unit 1b according to the present invention are characterized in that the opposing axes X and Y or the opposing axes X and Z of the two pairs of the opposed electrodes E₁₁ and E₁₂ and E₂₁ and E₂₂, respectively, are disposed so as to cross each other.

In the case of the detecting unit 1b, it is possible to form only one electrode E₂₁ of the second opposed electrodes E₂₁ and E₂₂ in the detecting unit 1b (the reaction area 2 of the detecting unit 1b) and use an external electrode that is not a component of the detecting unit 1b as the other electrode E₂₂. The external electrode (E₂₂) may be a fixed electrode formed on another substrate 4 or a movable electrode (not shown) capable of being moved to a position opposed to the electrode E₂₁ as required.

It is possible to employ a structure in which at least one electrode (for example the electrode E₂₁) or both the electrodes E₂₁ and E₂₂ of the second opposed electrodes E₂₁ and E₂₂ in the detecting unit 1a or the detecting unit 1b are formed by a conductor having optical transparency, such for example as ITO (Indium Tin Oxide) (structure of FIG. 2). Since electrodes that can transmit exciting light for detection can be formed by using this conductor having optical transparency, the conductor is suitable for use in detecting interaction in the reaction area 2 by optical means on the basis of a measurement of luminous intensity.

It is desirable that in order to facilitate generation of a non-uniform electric field in which lines of electric force are concentrated between the second opposed electrodes E₂₁ and E₂₂, the electrode E₂₁ forming the second opposed electrode be designed so as to have a smaller surface area than the other opposed electrode E₂₂ as shown in FIG. 2 and FIG. 3, for example.

Incidentally, as shown in FIG. 1, it is possible to adopt an embodiment in which a target substance T such as target DNA or the like is drawn to the vicinity of the electrode E₂₁ by making surface areas of the electrode E₂₁ and the electrode E₂₂ forming the second opposed electrodes equal to each other and forming a non-uniform electric field by concentrating lines of electric force on edges of the electrode E₂₁.

The electrode E₂₁ of the second opposed electrodes can be surface-treated in advance to fix a terminal of a detection substance D such as probe DNA or the like. As a method of fixing probe DNA as a detection substance D, for example, a terminal of the probe DNA may be fixed to a surface of the electrode by reaction such as coupling reaction or the like. For example, an electrode surface treated with streptavidin is suitable for fixing a biotinylated probe DNA terminal.

Alternatively, an electrode surface treated with a thiol (SH) radical is suitable for fixing probe DNA having a terminal modified by a thiol radical by a disulfide bond (—S—S— bond).

Thus, when a solution including a target substance T is injected into the reaction area 2 by dropping or the like in a state of the detection substance D such as probe DNA or the like being fixed to the surface of the electrode E₂₁ and the switch S₂ shown in the figures is turned on to apply a high-frequency alternating-current voltage between the second opposed electrodes E₂₁ and E₂₂ by the power supply V₂, a high-frequency alternating-current electric field can be formed in the reaction area 2. Incidentally, a high-frequency and high-voltage electric field of about 1×10⁶ V/m and about 1 MHz is suitable as the electric field applied between the second opposed electrodes E₂₁ and E₂₂ (see Masao Washizu and Osamu Kurosawa: “Electrostatic Manipulation of DNA in Microfabricated Structures,” IEEE Transaction on Industrial Application Vol. 26, No. 26, p. 1165 to 1172 (1990)).

When the high-frequency alternating-current electric field is formed in the reaction area 2, lines of electric force concentrate in the vicinity of the surface of the electrode E₂₁ having the smaller surface area or in the vicinity of the edges of the electrode E₂₁, whereby a non-uniform electric field is formed in the reaction area 2. The non-uniform electric field acts to stretch the target substance T such as target DNA or the like, which is present in the reaction area 2 in a randomly distributed state, along the non-uniform electric field, and thereby form the target substance T into a straight chain.

Further, the target substance T such as target DNA or the like can be moved (migrate) to the electrode E₂₁ having a higher field intensity in the non-uniform electric field generated in the reaction area 2 by electrodynamic force of dielectrophoresis. As a result, the target substance T such as target DNA or the like concentrates on the surface of the electrode E₂₁ on which the detection substance D such as probe DNA or the like is fixed in advance, so that an environment facilitating progress of interaction such as hybridization or the like can be formed.

Further, by making the target substance T such as target DNA or the like move (migrate) to the surface of the electrode E₂₁ in a short time by the effect of dielectrophoresis, and thus increasing concentration in a region near the surface of the electrode E₂₁, it is possible to reduce a time of interaction such as hybridization or the like between the target substance T such as target DNA or the like and the detection substance D such as probe DNA or the like.

Even if the non-uniform electric field is not generated between the second opposed electrodes E₂₁ and E₂₂, the target DNA present in the vicinity of the electrode E₂₁ electrically migrates to the surface of the electrode E₂₁ due to Coulomb force. As a result, the concentration of the target DNA increases in a field where hybridization progresses, so that the hybridization time can be reduced.

In addition, the elongating and aligning action of the electric field on the detection substance D such as probe DNA or the like can reduce hindrances to interaction such as hybridization or the like and false interaction such as mis-hybridization or the like that occur due to a steric hindrance caused by a higher order structure in a random coil shape of the detection substance D, for example.

When the interaction in the reaction area 2 is hybridization, a fluorescent intercalator injected into the reaction area 2 together with the target DNA in advance or a fluorescent intercalator injected after the hybridization emits fluorescent light in a double stranded nucleic acid formed by the hybridization, thus making it possible to detect the hybridization.

Incidentally, the hybridization can be detected by a method of detecting fluorescent light emitted by a fluorescent substance used for labeling the probe DNA as the detection substance D.

Next, a high-frequency alternating-current voltage is applied between the first opposed electrodes E₁₁ and E₁₂ by turning off the switch S₂ and turning on the switch S₁ shown in FIG. 1 and the like. Thereby a high-frequency alternating-current electric field is generated between the first opposed electrodes E₁₁and E₁₂, and in particular, a non-uniform electric field can be generated in the vicinity of edges of the electrodes E₁₁ and E₁₂.

As a result, surplus intercalators present in a free state in the detecting unit 1a or the detecting unit 1b and surplus target DNA that is not complementary and is in a free state can be moved in directions of strong electric fields, that is, in directions of edges of both the electrodes E₁₁ and E₁₂ along lines of electric force of the non-uniform electric field by effect of dielectrophoresis.

Further, the electric field generated between the first opposed electrodes E₁₁ and E₁₂ can forcefully dissociate target DNA that shows false interaction such as mis-hybridization or the like occurring on the surface of the electrode E₂₁ as a second opposed electrode (non-complementary target DNA) from the probe DNA as the fixed detection substance D, and draw the non-complementary target DNA to the electrode E₁₁ or the electrode E₁₂.

Thus, substances causing decrease in detection accuracy in the vicinity of the surface of the electrode E₂₁ as the second opposed electrode for providing a field of interaction such as hybridization or the like, for example surplus target DNA and surplus intercalators or substances showing false interaction such as mis-hybridization or the like can be moved (migrate) to the first opposed electrodes E₁₁ and E₁₂ having the opposing axis X crossing the opposing axis Y of the second opposed electrodes.

That is, the first opposed electrodes E₁₁ and E₁₂ perform a function of removing the substances causing decrease in detection accuracy from the field of interaction. This function makes it possible to obtain a detection signal with a reduced noise at the time of detection of interaction such as hybridization or the like. That is, a signal with a good S/N ratio can be obtained.

Incidentally, the present invention particularly uses the effects of applying an alternating-current electric field as means for removing surplus target DNA and surplus intercalators from the field for detecting hybridization. In general, since DNA is negatively charged and intercalators are positively charged, the surplus substances can be removed electrostatically by a direct-current electric field. However, the alternating-current electric field is suitable because the alternating-current electric field can be used to suppress heat generation and gas generation by electrolysis of an ionic solution in the vicinity of electrodes.

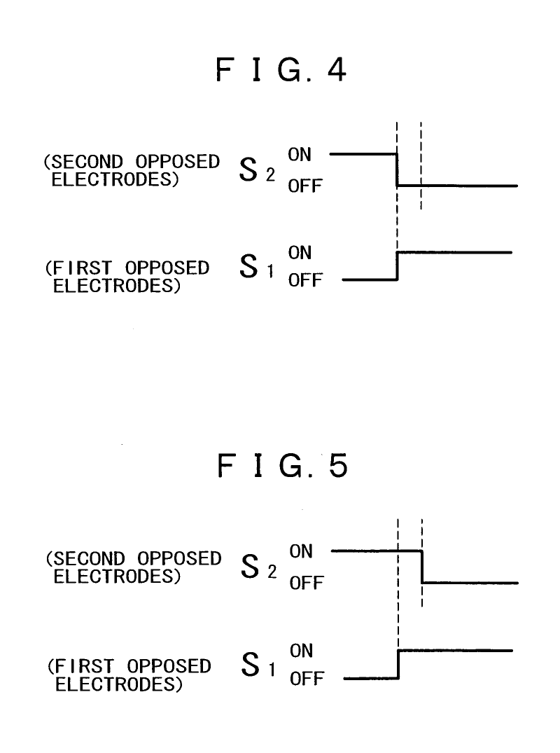

Description will now be made of timing of a procedure for turning on/off the switch S₁ and the switch S₂ with reference to FIG. 4 and FIG. 5. FIG. 4 is a diagram schematically showing an example of the procedure for turning on/off the switches. FIG. 5 is a diagram schematically showing a modified example of the procedure.

In the first example of FIG. 4, the switch S₁ for the first opposed electrodes E₁₁ and E₁₂ is turned on at the same time as the switch S₂ for the second opposed electrodes E₂₁ and E₂₂ is turned off. That is, application of voltage to the first opposed electrodes E₁₁ and E₁₂ that perform a function of drawing surplus substances thereto is started at the same time as application of voltage to the second opposed electrodes E₂₁ and E₂₂ that perform a function of elongating the fixed detection substance D and the like is stopped.

In the second modified example of FIG. 5, the switch S₁ for the first opposed electrodes E₁₁ and E₁₂ is turned on before the switch S₂ for the second opposed electrodes E₂₁ and E₂₂ is turned off. That is, a time is secured for which application of voltage to the first opposed electrodes E₁₁ and E₁₂ that perform the function of drawing surplus substances thereto and application of voltage to the second opposed electrodes E₂₁ and E₂₂ that perform the function of elongating the fixed detection substance D and the like are simultaneously performed.

It is desirable that interaction such as hybridization or the like be detected while maintaining a state in which surplus target DNA and surplus intercalators or substances showing false interaction such as mis-hybridization or the like (hereinafter referred to collectively as surplus substances) are drawn to the edges of the first opposed electrodes E₁₁ and E₁₂ with the alternating-current voltage applied between the first opposed electrodes E₁₁ and E₁₂. That is, in a stage of detection of interaction such as hybridization or the like, it is desirable that the detection of interaction such as hybridization or the like be performed with the switch S₁ for the first opposed electrodes E₁₁ and E₁₂ in an on state.

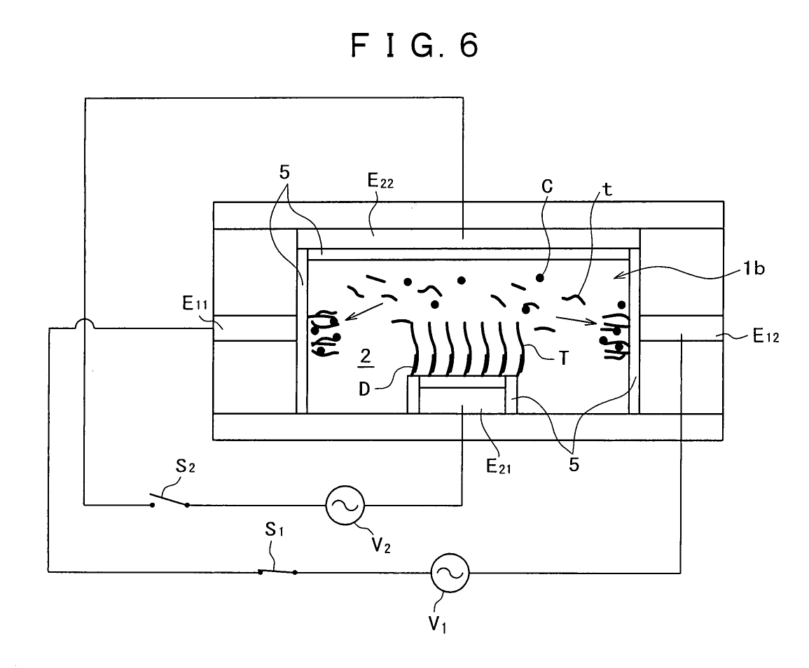

Taking the detecting unit 1b as an example, FIG. 6 schematically shows a state in which surplus target DNA denoted by reference character t and surplus intercalators denoted by reference character C are being drawn to the electrode E₁₁ and the electrode E₁₂ forming the first opposed electrodes while the application of voltage to the first opposed electrodes E₁₁ and E₁₂ is performed and the application of voltage to the second opposed electrodes E₂₁ and E₂₂ is stopped. Incidentally, reference numeral 5 in FIG. 6 specifically indicates an insulating layer (already described) formed by a material such as SiO₂, SiN, SiOC, SiC, SiOF, TiO₂ or the like for covering electrode surfaces.

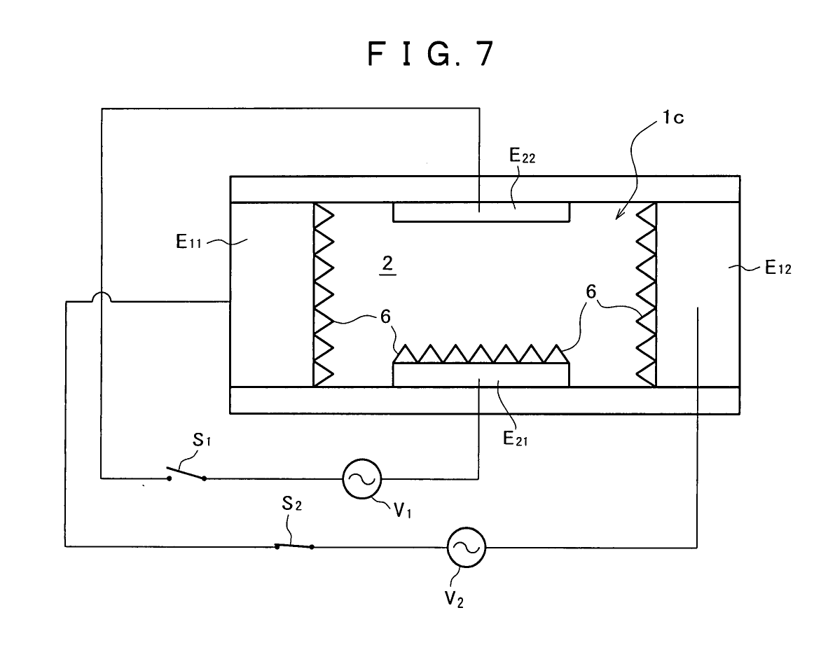

FIG. 7 is a diagram showing an example of a modified embodiment (reference character 1c) of a detecting unit according to the present invention. It is to be noted that FIG. 7 may be observed as a plan view (similar to FIG. 1) as viewed from above or a sectional view (similar to FIG. 2) taken in a vertical direction. Specifically, FIG. 7 includes both a structure in which opposing axes of two pairs of opposed electrodes cross each other on a plane (the structure is similar to that of the detecting unit 1a) and a structure in which second opposed electrodes E₂₁ and E₂₂ are disposed in a direction of a Z-axis (see FIG. 2) as in the detecting unit 1b.

As with the detecting units 1a and 1b, the detecting unit 1c is formed on for example a substrate formed of glass, synthetic resin, or the like. The detecting unit 1c is a part devised to detect interaction between substances. A reaction area 2 that can store a liquid phase serving as a reaction field or retain gel or the like similarly serving as a reaction field is also formed in the detecting unit 1c.

The detecting unit 1c is characterized in that electrode surfaces of first opposed electrodes E₁₁ and E₁₂ and a surface of one electrode E₂₁ forming a second opposed electrode are processed into a rough surface. Reference numeral 6 in FIG. 7 denotes a convex portion (mountain-shaped portion) formed by processing the surface into a rough surface, which portion is shown exaggerated. Incidentally, such electrode surfaces processed into rough surfaces can be adopted in other embodiments than the detecting unit 1c.

When the electrode surfaces of the first opposed electrodes E₁₁ and E₁₂ and the electrode E₂₁ are patterned with projections and depressions formed into an insular shape, for example, lines of electric force tend to concentrate on convex portions (mountain-shaped portions) 6 of the electrode surfaces, and thus non-uniform electric fields tend to be formed more easily. It is to be noted that the rough electrode surfaces are not limited to specific forms. Also, while a method of processing the electrode surfaces into rough surfaces can be carried out by using known sputtering techniques, etching techniques, and epitaxy techniques, for example, the rough surface processing method is not specifically limited.

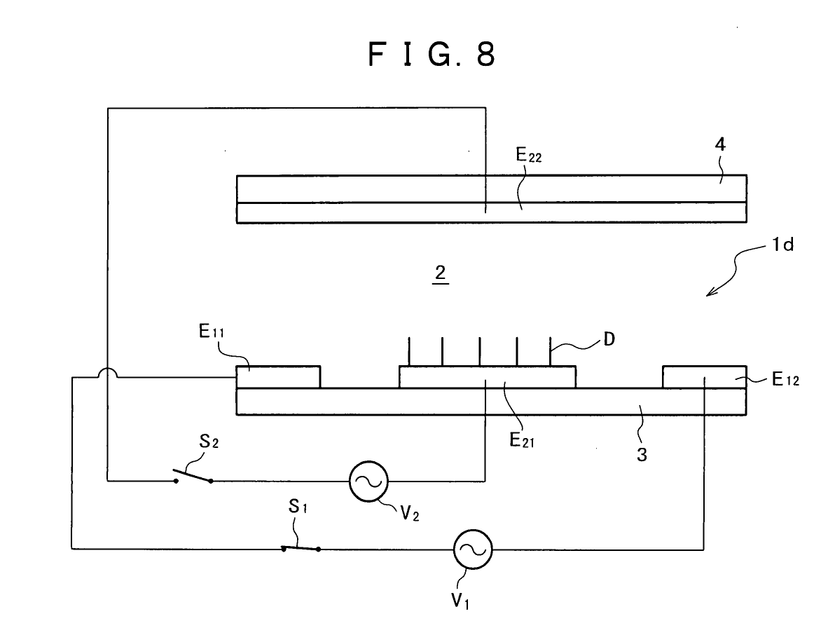

Other embodiments of the detecting unit according to the present invention will be described in the following with reference to FIGS. 8 to 10.

First, in a detecting unit 1d shown in FIG. 8, first opposed electrodes E₁₁ and E₁₂ for drawing and removing surplus substances from a field of interaction are on the same plane as an electrode E₂₁, on which a detection substance D such as probe DNA or the like is fixed, of second opposed electrodes E₂₁ and E₂₂ that perform functions of elongating and aligning the detection substance D by an electric field and the like (insulating layers 5 on the electrode surfaces are omitted). Thus, this example of the detecting unit is easier to fabricate. Reference numerals 3 and 4 denote a substrate. Incidentally, switches S₁ and S₂ in FIG. 8 may be turned on separately from each other, or may be turned on simultaneously.

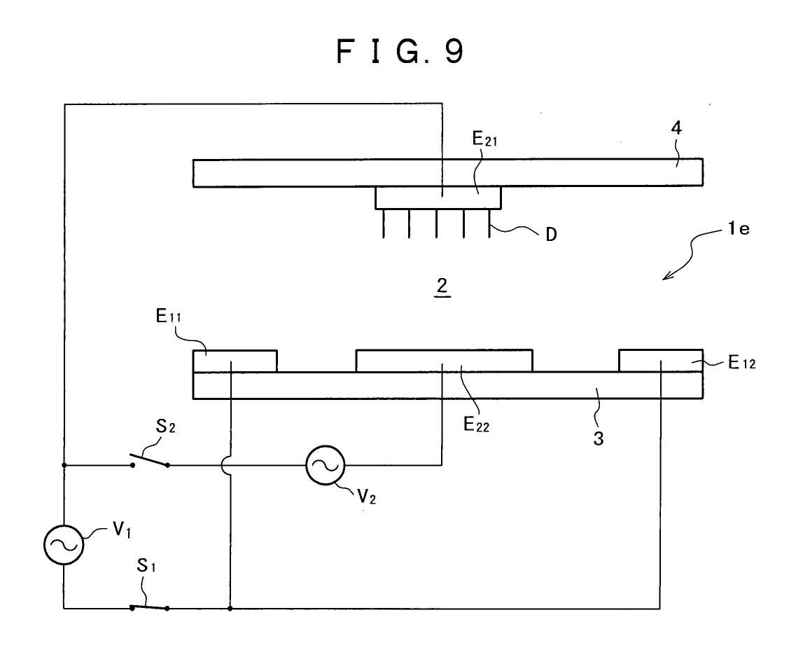

A detecting unit 1e shown in FIG. 9 is an embodiment in which the first opposed electrodes E₁₁ and E₁₂ are on the same plane as the electrode E₂₂, on which the detection substance D such as probe DNA or the like is not fixed, of the second opposed electrodes E₂₁ and E₂₂ (insulating layers 5 on the electrode surfaces are omitted). Also in this embodiment, the electrodes are easy to fabricate. Incidentally, in order to generate a non-uniform electric field in the vicinity of an electrode, it is desirable that the first opposed electrodes E₁₁ and E₁₂ have a smaller area than the electrode E₂₁.

In the detecting unit 1e, an alternating-current power supply V₁ can be connected to the first opposed electrodes E₁₁ and E₁₂ and the electrode E₂₁ by turning on a switch S₁, while an alternating-current power supply V₂ can be connected to the second opposed electrodes E₂₁ and E₂₂ by turning on a switch S₂. Incidentally, the switches S₁ and S₂ may be turned on separately from each other, or may be turned on simultaneously.

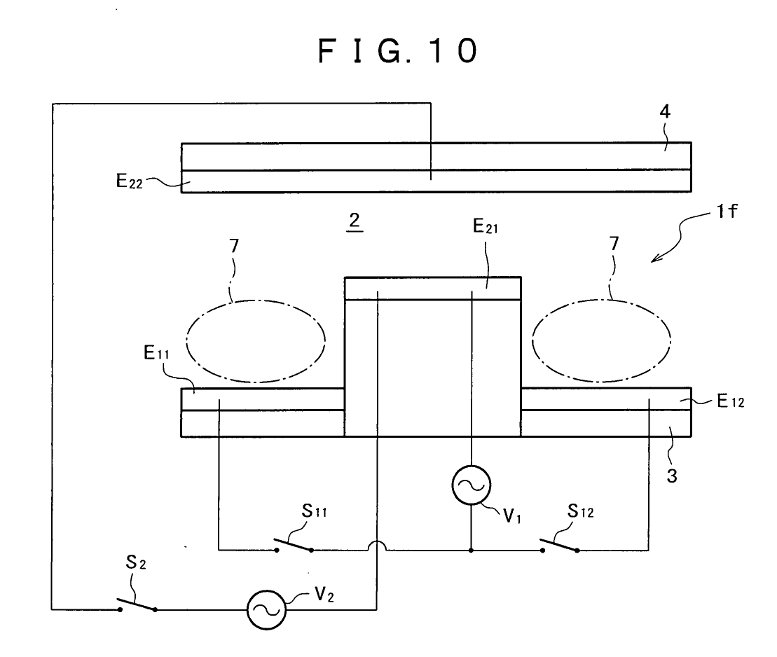

A detecting unit 1f in FIG. 10 is an embodiment in which the first opposed electrodes E₁₁ and E₁₂ are disposed at a position lower than that of the electrode E₂₁, on which the detection substance D such as probe DNA or the like is fixed, of the electrode pair of second opposed electrodes E₂₁ and E₂₂.

In the case of the detecting unit 1f, surplus substances drawn to the first opposed electrodes E₁₁ and E₁₂ stay in regions indicated by reference numeral 7 in FIG. 10. Therefore, unlike the detecting units 1a and 1b, there is no particular need for maintaining a state of voltage being applied to the first opposed electrodes E₁₁ and E₁₂ at a time of detection, for example.

The detecting unit 1f is devised so as to apply a voltage between the electrode E₁₁ and the electrode E₂₁ by turning on only a switch S₁₁, apply the voltage between the electrode E₁₂ and the electrode E₂₁ by turning on only a switch S₁₂, and apply the voltage both between the electrode E₁₁ and the electrode E₂₁ and between the electrode E₁₂ and the electrode E₂₁ by turning on both the switches S₁₁ and S₁₂ (see FIG. 10). Incidentally, the switches S₁₁ and S₁₂ and a switch S₂ may be turned on separately from each other, or may be turned on simultaneously.

In addition, though not specifically shown, an embodiment can be adopted in which the first opposed electrodes E₁₁ and E₁₂ are disposed at a position lower than that of the electrode E₂₂, on which the detection substance D such as probe DNA or the like is not fixed, of the electrode pair of second opposed electrodes E₂₁ and E₂₂.

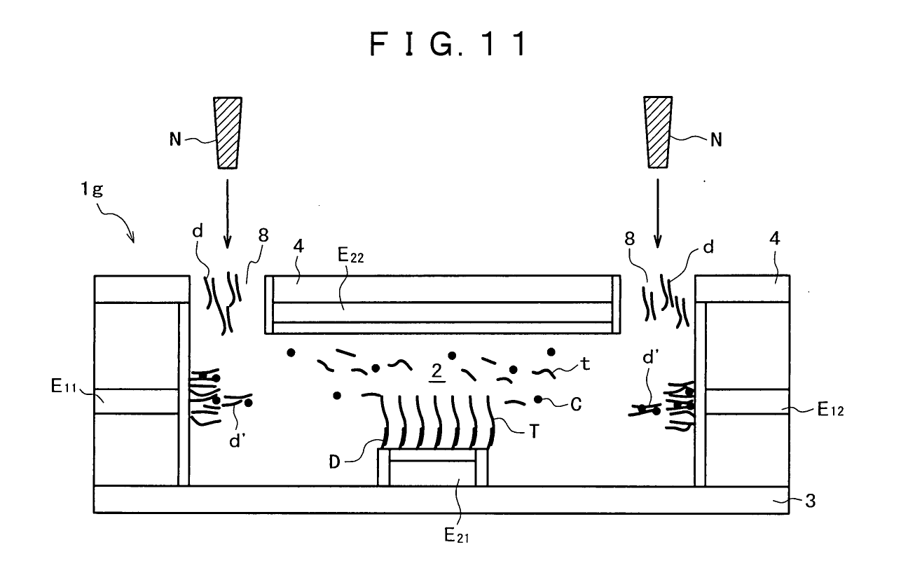

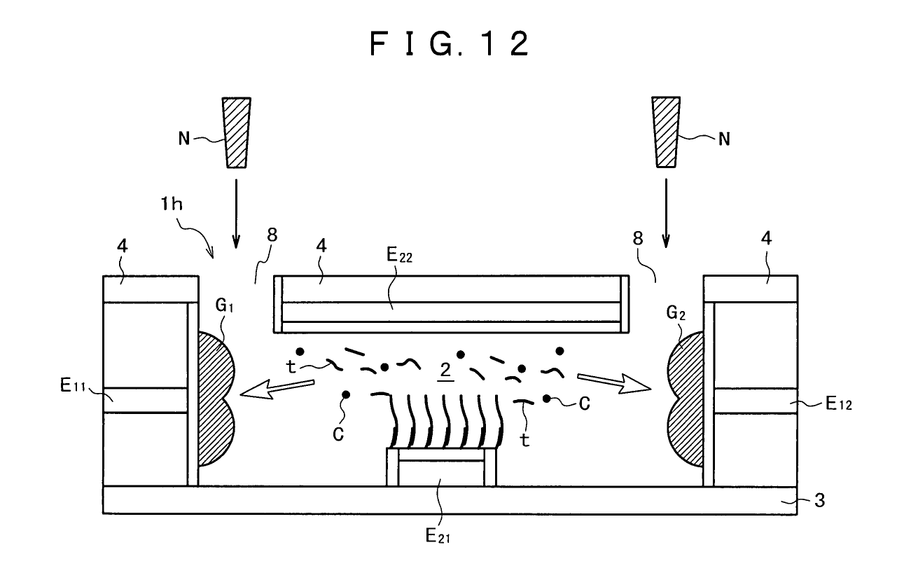

Next, developed embodiments of the detecting unit according to the present invention will be described with reference to FIG. 11 and FIG. 12.

When probe DNA having a terminal thereof biotinylated is to be fixed on a streptavidin-treated surface of an electrode E₂₁ as a second opposed electrode, for example, surfaces of first opposed electrodes E₁₁ and E₁₂ are also treated with streptavidin.

Next, when target DNA as a target substance T having a terminal thereof treated with biotin is added to a reaction area 2, hybridization is performed between the target DNA and the probe DNA under an electric field applied between the second opposed electrodes E₂₁ and E₂₂. Thereafter surplus target DNA denoted by reference character t which DNA is drawn to the vicinity of the surfaces of the first opposed electrodes E₁₁ and E₁₂ by effect of dielectrophoresis is fixed and trapped on each of the electrode surfaces of the first opposed electrodes E₁₁ and E₁₂ by an avidin-biotin bond.

Then, after the hybridization, as in a detecting unit 1g shown in FIG. 11, “dummy double stranded DNA” denoted by reference character d and having a terminal thereof biotinylated is dropped or injected from nozzles N into regions near the first opposed electrodes E₁₁ and E₁₂. Thereby, surplus intercalators C being drawn to the vicinity of the surfaces of the first opposed electrodes E₁₁ and E₁₂ by dielectrophoresis can be stably trapped within the dummy double stranded DNA, and the dummy double stranded DNA can be fixed and trapped on the surfaces of the first opposed electrodes E₁₁ and E₁₂ by the avidin-biotin bond.

Thus, the detecting unit 1g of the embodiment as shown in FIG. 11 can fix and retain the surplus target DNA and the surplus intercalators C on the surfaces of the first opposed electrodes E₁₁ and E₁₂.

Unlike the detecting unit 1a, the detecting unit 1b or the like, such an embodiment eliminates the need for detecting a hybridization signal while maintaining a state in which an electric field is applied between the first opposed electrodes E₁₁ and E₁₂ and surplus substances are drawn to the first opposed electrodes E₁₁ and E₁₂ by effect of the electric field. Incidentally, instead of the avidin-biotin bond, a disulfide bond may be employed using a thiol (SH) radical.

Dispensers, ink jet nozzles and the like can be used for dropping or injecting the “dummy double stranded DNA” denoted by reference character d in FIG. 11. Opening portions 8 in communication with the reaction area 2 are formed in the detecting unit to enable the dropping or injecting of the dummy double stranded DNA (this point is common to all the embodiments). Incidentally, reference character d′ in FIG. 11 indicates a state in which surplus intercalators C are trapped in dummy double stranded DNAs.

Another means for trapping surplus target DNA as a free substance will be described. First, a single stranded nucleic acid (not shown) having a base sequence complementary to a base sequence specific to surplus target DNA denoted by reference character t or a base sequence modified at a terminal of the target DNA is made to be present on surfaces of electrodes E₁₁ and E₁₂ forming first opposed electrodes by dropping or the like. Next, the surplus target DNA is trapped on the surfaces of the electrodes E₁₁ and E₁₂ by hybridization between the single stranded nucleic acid and the surplus target DNA.

Then, after the hybridization in a reaction area 2, as in a detecting unit 1h shown in FIG. 12, gel G₁ including cations and gel G₂ including anions are dropped or injected from nozzles N through opening portions 8 into regions in the vicinity of the first opposed electrodes E₁₁ and E₁₂.

Positively charged surplus intercalators C drawn by dielectrophoresis to the region in the vicinity of the first opposed electrodes E₁₁ and E₁₂ to which voltage is applied are electrostatically bonded to the cations in the gel G₁ and thereby trapped, and negatively charged surplus DNA denoted by reference character t is electrostatically bonded to the anions in the gel G₂ and thereby trapped.

Thus, the detecting unit 1h shown in FIG. 12 can also fix and retain surplus DNA and surplus intercalators on the electrode surfaces of the first opposed electrodes E₁₁ and E₁₂. Unlike the detecting unit 1a, the detecting unit 1b or the like, the detecting unit 1h eliminates the need for detecting a hybridization signal while maintaining a state in which an electric field is applied between the first opposed electrodes E₁₁ and E₁₂ and the surplus substances are drawn to the first opposed electrodes E₁₁ and E₁₂ by effect of the electric field.

Incidentally, materials for the gel G₁ including cations include for example a material having —COO⁻ attached to a polymer chain, and materials for the gel G₂ including anions include for example a material having —NH³⁺ attached to a polymer chain.

In another modified embodiment, the gel G may be a neutral gel in which 20 to 30 mer DNA having an entire base sequence including (T) thymine (hereinafter described as Poly-T), for example, is mixed, and target DNA may be modified with a 20 to 30 mer base sequence all of which includes A (adenine) (hereinafter described as Poly-A).

Poly-A added to surplus DNA drawn to the neutral gel (not shown) near first opposed electrodes E₁₁ and E₁₂ by dielectrophoresis and Poly-T present in the gel are bonded to each other by hybridization. Thus the surplus DNA is trapped in the neutral gel.

Further, surplus intercalators C drawn to the vicinity of the first opposed electrodes E₁₁ and E₁₂ by dielectrophoresis are introduced into a complementary chain bond body of Poly-A and Poly-T generated by the hybridization, and are thus trapped in the neutral gel together with the surplus DNA.

Thus, such an embodiment can also fix and retain surplus DNA and surplus intercalators in the vicinity of the first opposed electrodes E₁₁ and E₁₂. Unlike the detecting unit 1a, the detecting unit 1b or the like, the embodiment eliminates the need for detecting a hybridization signal while maintaining a state in which an electric field is applied between the first opposed electrodes E₁₁ and E₁₂ and the surplus substances are drawn to the first opposed electrodes E₁₁ and E₁₂ by effect of the electric field.

As described above, the present invention can suitably employ a structure in which electrode surfaces are covered with an insulating layer such as SiO₂ or the like to prevent electrochemical reaction. A structure is devised so as to form an insulating layer using titanium dioxide (TiO₂) in place of the SiO₂.

In such a structure, after surplus intercalators and surplus DNA are drawn to and collected in the vicinity of the first opposed electrodes E₁₁ and E₁₂ by dielectrophoresis, the insulating layer formed of the TiO₂ is irradiated with ultraviolet light of 380 nm or less. Incidentally, it is desirable that in order to reliably irradiate the TiO₂ with ultraviolet light, electrodes other than the electrode E₂₁ be optically transparent electrodes such for example as ITO.

TiO₂ becomes a catalyst by being irradiated with ultraviolet light, and thus decomposes organic matter on a surface thereof into H₂O and CO₂ by oxidation-reduction reaction. Hence, surplus intercalators and surplus DNA, which are organic matter, can be decomposed. Thus, surplus intercalators and surplus DNA that can cause noise in a hybridization signal can be removed from the reaction area 2.

By disposing the detecting units 1a to 1g described above in a predetermined arrangement on a substrate, it is possible to provide a bioassay substrate such as a DNA chip or the like that can make interaction such as hybridization or the like progress in a short time and enables comprehensive analysis.

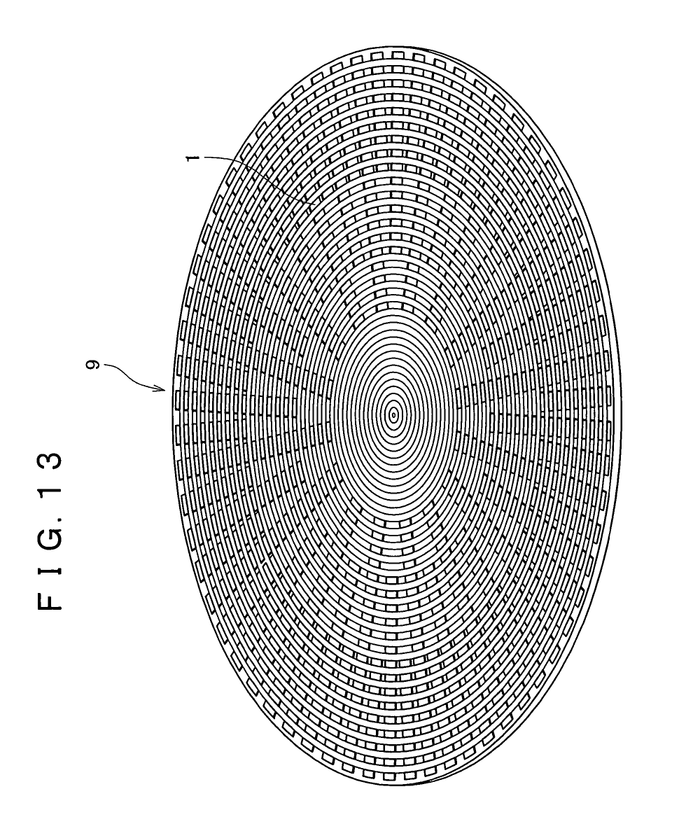

FIG. 13 is a diagram showing an example of the bioassay substrate. As shown in FIG. 13, a large number of detecting units can be disposed on a substrate 9 in a form of a disc so as to be able to be divided into groups. Reference numeral 1 in FIG. 13 denotes one of the embodiments of the detecting unit according to the present invention.

Incidentally, interaction that has progressed in one of the detecting units 1 disposed on the substrate 9 can be detected by known optical detecting means for irradiating for example a fluorescent substance attached in advance as a label to a detection substance D fixed on the surface of an electrode E₂₁ forming a second opposed electrode or a fluorescent intercalator inserted into and bonded to a substance (double stranded nucleic acid) showing interaction with fluorescence exciting light of a predetermined wavelength, and detecting the result.

The detecting unit according to the present invention can greatly reduce a time of interaction such as hybridization or the like in the detecting unit because of good efficiency of the interaction, and reduce occurrence of false positives and false negatives because the detecting unit can form environment that facilitates progress of correct interaction. Thus, the detecting unit can be used for a bioassay substrate such as a DNA chip or the like having characteristics of providing greater efficiency of assay work for detecting interaction and high detection accuracy.

While the preferred embodiments of the present invention have been described using specific embodiments, such description is for illustrative purposes only, and it is to be understood that changes and variations may be made without departing from the spirit or scope of the appended claims.

1. A detecting unit for detecting interaction between substances, said detecting unit comprising:
a pair of first opposed electrodes disposed opposite to each other so as to sandwich a reaction area providing a field for the interaction between the substances; and
both or one of electrodes forming second opposed electrodes disposed opposite to each other in a direction of an axis crossing an opposing axis of said first opposed electrodes.

2. A detecting unit for detecting interaction between substances, as claimed in claim 1,
wherein the opposing axis of said second opposed electrodes is perpendicular to a surface on which a substance for detection is fixed in said reaction area.

3. A detecting unit for detecting interaction between substances, as claimed in claim 1,
wherein said first opposed electrodes are means for drawing free substance and/or substance showing false interaction that are present in said reaction area to each electrode side by an electric field.

4. A detecting unit for detecting interaction between substances, as claimed in claim 1,
wherein said second opposed electrodes are means for elongating a substance for detection fixed in said reaction area by an electric field.

5. A detecting unit for detecting interaction between substances, as claimed in claim 1,
wherein said first opposed electrodes are means for drawing free substance and/or substance showing false interaction that are present in said reaction area to each electrode side by an electric field; and
said second opposed electrodes are means for elongating a substance for detection fixed in said reaction area by an electric field.

6. A detecting unit for detecting interaction between substances, as claimed in claim 1,
wherein said first opposed electrodes and said second opposed electrodes are electrodes for application of an alternating-current electric field.

7. A detecting unit for detecting interaction between substances, as claimed in claim 1,
wherein an area of one of said second opposed electrodes is smaller than an area of the other opposite electrode.

8. A detecting unit for detecting interaction between substances, as claimed in claim 1,
wherein a surface of at least one of said second opposed electrodes is formed into a rough surface.

9. A detecting unit for detecting interaction between substances, as claimed in claim 1,
wherein at least one of said second opposed electrodes is formed by patterning.

10. A detecting unit for detecting interaction between substances, as claimed in claim 1,
wherein said interaction is hybridization.

11. A detecting unit for detecting interaction between substances, as claimed in claim 1,
wherein surfaces of said electrodes are covered with an insulating layer.

12. A detecting unit for detecting interaction between substances, as claimed in claim 9,
wherein said insulating layer is formed by a material selected from SiO₂, SiN, SiOC, SiC, SiOF, and TiO₂.

13. A detecting unit for detecting interaction between substances, as claimed in claim 2,
wherein at least one of said second opposed electrodes is formed by a transparent conductor.

14. A detecting unit for detecting interaction between substances, as claimed in claim 3,
wherein nucleic acid as substance drawn to electrodes forming said first opposed electrodes is fixed on surfaces of said electrodes via one of an avidin-biotin bond and a disulfide bond.

15. A detecting unit for detecting interaction between substances, as claimed in claim 3,
wherein a surplus intercalator as substance drawn to electrodes forming said first opposed electrodes is trapped by a double stranded nucleic acid added in a vicinity of surfaces of said electrodes; and
said double stranded nucleic acid is trapped on the surfaces of said electrodes via one of an avidin-biotin bond and a disulfide bond.

16. A detecting unit for detecting interaction between substances, as claimed in claim 3,
wherein a gel including cations and a gel including anions are made to be present in advance on surfaces of electrodes forming said first opposed electrodes; and
a negatively charged nucleic acid and a positively charged surplus intercalator as substances drawn to each of said first opposed electrodes are electrostatically bonded to said cations and said anions, respectively.

17. A detecting unit for detecting interaction between substances, as claimed in claim 3,
wherein said detecting unit is a detecting unit for detecting hybridization between a nucleic acid for detection and a target nucleic acid present in said reaction area;
a single stranded nucleic acid having a base sequence complementary to one of a base sequence specific to said target nucleic acid and a base sequence modified at a terminal of said target nucleic acid is made to be present on surfaces of electrodes forming said first opposed electrodes; and
surplus target nucleic acid is trapped on the surfaces of said electrodes by hybridization between said single stranded nucleic acid and said surplus target nucleic acid.

18. A detecting unit for detecting interaction between substances, as claimed in claim 17,
wherein a surplus intercalator is trapped in one of a double stranded nucleic acid obtained by said hybridization and a double stranded nucleic acid added separately.

19. A detecting unit for detecting interaction between substances, as claimed in claim 3,
wherein substance drawn to electrodes forming said first opposed electrodes is decomposed by covering surfaces of the electrodes forming said first opposed electrodes with an insulating layer formed of TiO₂, irradiating said insulating layer with ultraviolet light, and making said TiO₂ function as a catalyst.

20. A bioassay substrate having the detecting unit for detecting interaction between substances as claimed in claim 1.

21. A bioassay substrate as claimed in claim 20,
wherein said interaction is hybridization.

22. A hybridization detecting unit comprising at least:
opposed electrodes for elongating nucleic acid as a complementary chain for hybridization; and
an electrode used for removing free nucleic acid present in a field of said hybridization and/or nucleic acid showing mis-hybridization.

23. A DNA chip having the detecting unit as claimed in claim 22.
